(19) United States
(12) Patent Application Publication
(10) Pub. No.: US 2012/0128764 A1
(43) Pub. Date: May 24, 2012

(54) CONTROLLED-RELEASE COMPOSITIONS COMPRISING A PROTON PUMP INHIBITOR

(75) Inventors: Gopi Venkatesh, Vandalia, OH (US); Michael Gosselin, Springboro, OH (US); Jin-Wang Lai, Springboro, OH (US); Christian Stollberg, Carugate (IT); Flavio Fabiani, Merate-LC (IT)

(73) Assignee: Aptalis Pharmatech, Inc., Vandalia, OH (US)

(21) Appl. No.: 13/202,959

(22) PCT Filed: Feb. 23, 2010
(86) PCT No.: PCT/US2010/025067

§ 371 Date: Feb. 6, 2012

Related U.S. Application Data
(60) Provisional application No. 61/154,506, filed on Feb. 23, 2009.

(51) Int. Cl. A61K 9/48; B05D 5/00; A61K 31/4439; A61K 9/20; A61K 9/16

(52) U.S. Cl. 424/451; 424/464; 424/490; 424/494; 424/497; 514/338; 427/2.21

(57) ABSTRACT

The present invention relates to pharmaceutical compositions, and methods of preparing such compositions, comprising one or more populations of controlled-release particles comprising one or more proton pump inhibitors. The present invention also relates to pharmaceutical dosage forms, including orally disintegrating tablets, tablets, capsules, and methods for their preparation.

### CROSS REFERENCE TO RELATED APPLICATIONS

The present application claims priority to U.S. Provisional Application No. 61/154,506 filed Feb. 23, 2009, which is incorporated herein by reference in its entirety for all purposes.

### BACKGROUND OF THE INVENTION

Proton pump inhibitors (PPIs) are highly effective gastric secretion inhibitors. They are a group of acid-unstable physiologically active antisecretory compounds that do not exhibit anticholinergic or histamine H₂-receptor antagonist properties. Examples of PPIs include omeprazole, esomeprazole, lansoprazole, rabeprazole, pantoprazole, pariprazole, lemiprazole, tenatoprazole, nepaprazole, and ilaprazole. Worldwide clinical experience with PPIs in recent years has established their effectiveness in treating acid reflux-related diseases, including gastric and duodenal ulcers, gastroesophageal reflux disease (GERD), and even erosive esophagitis. In addition, PPIs are well tolerated, and the few serious side effects reported (e.g., diarrhea, abdominal pain and nausea) occur infrequently.

Extended-release dosage forms for once-daily oral administration of PPIs are available, though such dosage forms do not effectively control gastroesophageal symptoms over the full course of a day. Due to the short plasma half-lives of PPIs, a single “pulse” or release of a PPI from a dosage form (even extended release) typically does not provide relief over 24 hours. This shortcoming of currently available PPI dosage forms allows gastro-esophageal symptoms to “breakthrough” when PPI plasma levels drop below therapeutic range. Breakthrough tends to occur at night, resulting in nocturnal acid breakthrough (NAB), a common symptom among GERD sufferers taking PPIs to manage the disease. In fact, NAB occurs in 70% of GERD patients. NAB is a particularly worrisome symptom because, due to the supine position of patients at night, it can cause prolonged exposure of the esophagus to acid (i.e., acid reflux) and can eventually lead to erosive esophagitis. Esophageal acid reflux has been observed in 33% of those patients suffering from NAB. Thus, it is desirable to provide a formulation which achieves adequate PPI plasma concentrations sufficient to inhibit proton pumps when they become active in the middle of the night.

A dosage form that provided bimodal release (or a second “pulse”) to boost waning PPI plasma levels would better treat the symptoms of GERD sufferers. However, technical challenges hinder the development such bimodal-release dosage forms for PPIs. For example, PPIs are acid-sensitive, rapidly absorbed with mean plasma levels occurring after about 1.5-2.5 hrs, and have a plasma half-life of about 1.5±1 hrs. Thus, an orally administered once-a-day PPI formulation would ideally: protect the PPI against degradation by stomach acids; simultaneously provide a relatively rapid release of PPI for short-term relief of GERD symptoms; sufficiently retard release of the PPI to provide therapeutic levels of the PPI over time; and provide a second “pulse” of PPI sufficient to prevent NAB. Reconciling these multiple technical requirements is difficult.

Furthermore, even if a fully effective bimodal-release, once-daily dosage form for PPIs existed, it still might not serve the needs of patients who have difficulty ingesting conventional dosage forms, due to dysphagia or impaired swallowing. For example, many elderly patients have higher frequencies of dysphagia and also suffer from acid reflux-related disorders. Therefore, an extended-release PPI dosage form that affords a more convenient mode of oral administration would be a welcome alternative for those patients who cannot or prefer not to swallow conventional dosage forms.

Orally disintegrating tablets (ODTs) offer a preferable alternative for patients with dysphagia, but ODTs are difficult to formulate as extended-release dosage forms. ODTs rapidly disintegrate on contact with the saliva in the oral cavity without the need for water. Furthermore, in order to enhance patient compliance, ODTs must exhibit acceptable organoleptic properties: i.e., a smooth “mouthfeel” achieved through smaller particle size and acceptable taste properties, while also providing acceptable pharmacokinetic properties appropriate for the condition treated (e.g., bimodal release profile with Cₘₐₓ of the pulses separated by 1-6 hours). Simultaneously achieving acceptable organoleptic and controlled-release properties for PPIs is challenging for several reasons. First, because PPIs are extremely acid sensitive, PPI formulations are typically stabilized with strong alkaline agents, and therefore a thicker polymer coating with an enterosoluble polymer is typically used to prevent degradation of the PPI in the stomach. However, thicker coatings provide relatively large particle sizes that can create a “gritty” mouthfeel, and therefore compromise organoleptic properties. Second, treatment of nocturnal symptoms (e.g., NAB) requires an extended lag times before release of a second “pulse” of PPI, requiring thicker and/or additional coating(s), which, again, can compromise organoleptic properties.

### SUMMARY OF THE INVENTION

In one embodiment, the present invention relates to a pharmaceutical composition comprising a first population of controlled-release particles, wherein the controlled-release particles of the first population comprise: a core comprising a proton pump inhibitor or a pharmaceutically acceptable salt, solvate, and/or ester thereof, and an alkaline agent; a first coating disposed over said core, comprising an enteric polymer, and a second coating disposed over the core, comprising an enteric polymer and a water-insoluble polymer, wherein the first coating is substantially free of water-insoluble polymers.

In another embodiment, the present invention relates to a pharmaceutical composition comprising the first population of controlled-release particles described herein, and further comprising a second population of controlled-release particles, wherein the controlled-release particles of the second population comprise: a core comprising a proton pump inhibitor or a pharmaceutically acceptable salt, solvate, and/or ester thereof; and at least one controlled-release coating disposed over said core, comprising an enteric polymer.

In one embodiment, the present invention relates to a pharmaceutical dosage form comprising: (i) a first population of controlled-release particles, wherein the controlled-release particles of the first population comprise: a core comprising a proton pump inhibitor or a pharmaceutically acceptable salt, solvate, and/or ester thereof; a first coating disposed over the core, comprising an enteric polymer; and a second coating disposed over the core, comprising an enteric polymer and a water-insoluble polymer, wherein the first coating is substantially free of water-insoluble polymers; and (ii) rapidly dispersing granules comprising a saccharide and/or sugar alcohol in combination with a disintegrant.

In another embodiment, the present invention relates to a pharmaceutical dosage form comprising: (i) the first population of controlled-release particles, as described herein; (ii) a second population of controlled-release particles, comprising a core comprising a proton pump inhibitor or a pharmaceutically acceptable salt, solvate, and/or ester thereof; and (iii) rapidly dispersing granules comprising a saccharide and/or sugar alcohol in combination with a disintegrant.

In one embodiment, the present invention relates to a method of preparing a first population of controlled-release particles, as described herein, comprising: preparing a core comprising said proton pump inhibitor or a pharmaceutically acceptable salt, hydrate, polymorph, solvate, and/or ester thereof; disposing a delayed release coating comprising an enteric polymer over the core; and disposing a timed, pulsatile-release coating comprising an enteric polymer in combination with a water-insoluble polymer over the core.

In another embodiment, the present invention relates to a method of preparing an orally disintegrating tablet comprising mixing the first population of controlled-release particles, as described herein, with rapidly dispersing granules comprising a saccharide and/or sugar alcohol in combination with a disintegrant, thereby forming a compressible blend; and compressing the compressible blend into an orally disintegrating tablet.

In yet another embodiment, the present invention relates to a method of preparing an orally disintegrating tablet comprising: (i) mixing the first population of controlled-release particles, as described herein, with the second population of controlled-release particles, as described herein, and rapidly dispersing granules comprising a saccharide and/or sugar alcohol in combination with a disintegrant, thereby forming a compressible blend; and (ii) compressing the compressible blend into an orally disintegrating tablet.

### BRIEF DESCRIPTION OF THE DRAWINGS

FIG. 1.A illustrates the cross-section of one embodiment of a delayed-release bead 10 comprising an inert core 2, a drug layer 4 comprising a proton pump inhibitor, a sealant layer 6, and an enteric polymer layer 8.

FIG. 1.B illustrates the cross-section of another embodiment of a controlled-release bead 20 comprising an inert core 2, a drug layer 4 comprising a proton pump inhibitor, a sealant layer 6, an inner coating 16 comprising a water-insoluble polymer in combination with an enteric polymer, and an outer coating comprising an enteric polymer 18. Alternatively, in FIG. 1.B, the inner coating 16 may comprise an enteric polymer while the outer coating layer 18 may comprise a water-insoluble polymer in combination with an enteric polymer.

FIG. 2 illustrates the in vitro pantoprazole release profiles of timed, pulsatile-release beads of Example 1.

FIG. 3 illustrates the in vitro pantoprazole release profiles of controlled-release beads of Example 2.A.

FIG. 4 illustrates the in vitro pantoprazole release profiles of controlled-release beads of Example 2.B.

FIG. 5 illustrates the in vitro pantoprazole release profiles of controlled-release beads of Example 3.

FIG. 6 illustrates the in vitro pantoprazole release profiles of controlled-release beads of Examples 2 and 3.

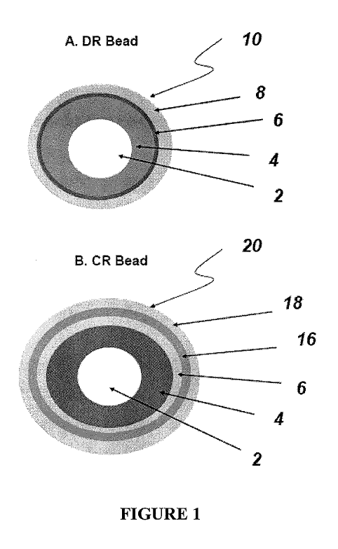

### DETAILED DESCRIPTION OF THE INVENTION

All documents cited are incorporated herein by reference in their entirety for all purposes to the same extent as if each individual document was specifically and individually indicated to be incorporated by reference. The citation of any document is not to be construed as an admission that it is prior art with respect to the present invention.

The terms “drug,” “active” and “active pharmaceutical ingredient” as used herein include a pharmaceutically acceptable and therapeutically effective compound, pharmaceutically acceptable salts, stereoisomers and mixtures of stereoisomers, solvates (including hydrates), polymorphs, and/or esters thereof. When referring to a drug (e.g., a PPI) in the descriptions of the various embodiments of the invention, the reference encompasses the base drug, pharmaceutically acceptable salts, stereoisomers and mixtures of stereoisomers, solvates (including hydrates), polymorphs, and/or esters thereof.

The term “salts” refers to the product formed by the reaction of a suitable inorganic or organic acid with the “free base” form of the drug. Suitable acids include those having sufficient acidity to form a stable salt, for example acids with low toxicity, such as the salts approved for use in humans or animals. Non-limiting examples of acids which may be used to form salts of dicyclomine include inorganic acids, e.g., HF, HCl, HBr, H₁, H₂SO₄, H₃PO₄; non-limiting examples of organic acids include organic sulfonic acids, such as C₆₋₁₆ aryl sulfonic acids, C₆₋₁₆ heteroaryl sulfonic acids or C₁₋₁₆ alkyl sulfonic acids—e.g., phenyl, a-naphthyl, β-naphthyl, (S)-camphor, methyl, ethyl, n-propyl, i-propyl, n-butyl, s-butyl, i-butyl, t-butyl, pentyl and hexyl sulfonic acids; non-limiting examples of organic acids includes carboxylic acids such as C₁₋₁₆ alkyl, C₆₋₁₆ aryl carboxylic acids and C₄₋₁₆ heteroaryl carboxylic acids, e.g., acetic, glycolic, lactic, pyruvic, malonic, glutaric, tartaric, citric, fumaric, succinic, malic, maleic, hydroxymaleic, benzoic, hydroxybenzoic, phenylacetic, cinnamic, salicylic and 2-phenoxybenzoic acids; non-limiting examples of organic acids include amino acids, e.g. the naturally-occurring amino acids, lysine, arginine, glutamic acid, glycine, serine, threonine, alanine, isoleucine, leucine, etc. Other suitable salts can be found in, e.g., S. M. Birge et al., J. Pharm. Sci., 1977, 66, pp. 1-19 (herein incorporated by reference for all purposes). In most embodiments, “salts” refers to salts which are biologically compatible or pharmaceutically acceptable or non-toxic, particularly for mammalian cells. The salts of drugs useful in the present invention may be crystalline or amorphous, or mixtures of different crystalline forms and/or mixtures of crystalline and amorphous forms.

The terms “orally disintegrating tablet” or “ODT” refers to a tablet which disintegrates rapidly in the oral cavity of a patient after administration, without the need for chewing. The rate of disintegration can vary, but is faster than the rate of disintegration of conventional solid dosage forms (e.g., tablets or capsules) which are intended to be swallowed immediately after administration, or faster than the rate of disintegration of chewable solid dosage forms, when tested as described herein (e.g. the USP <701> test method).

The term “about” is used herein to refer to a numerical quantity, and includes “exactly.” For example, “about 60 seconds” includes 60 seconds, exactly, as well as values close to 60 seconds (e.g., 50 seconds, 55 seconds, 59 seconds, 61 seconds, 65 seconds, 70 seconds, etc.).

The term “core” includes but is not limited to a bead, pellet, microgranule, granulate, mini-tablet, drug crystal, etc., having a size typically in the range of from about 100 μm to about 2 mm including from about 1000 μm to about 1500 μm, about 800 μm to about 1200 μm, about 100 μm to about 1000 μm, about 100 μm to about 800 μm, about 100 μm to about 600 μm, about 100 μm to about 500 μm, about 100 μm to about 1400 μm, about 200 μm to about 500 μm, about 200 μm to about 800 μm, about 300 μm to about 400 μm, about 300 μm to about 500 μm, about 300 μm to about 600 μm, and all subranges therebetween.

As used herein, the term “controlled release” coating encompasses coatings that delay release, sustain release, extend release, prevent release, and/or otherwise prolong the release of a drug relative to formulations lacking such coatings which release a drug relatively quickly (i.e., “immediate release” compositions). The term “controlled release” encompasses “sustained release,” “extended release,” “delayed release,” and “timed, pulsatile release.” The term “lag-time” coating refers to a particular type of “controlled release” coating in which the lag time coating delays release of a drug after administration. The term “controlled release” is used interchangeably with “modified release.” The term “controlled-release bead” or “controlled-release particle” refers broadly to a bead or particle showing one or more controlled-release properties, as described herein. The term “controlled-release bead” or “controlled-release particle” also refers to a drug-containing particle coated with one or more controlled-release coatings, as described herein.

The term “pH sensitive” as used herein refers to polymers which exhibit pH dependent solubility.

The term “enteric polymer,” as used herein, refers to a pH sensitive polymer that is resistant to gastric juice (i.e., relatively insoluble at the low pH levels found in the stomach), and which dissolves at the higher pH levels found in the intestinal tract.

The term “lag time” refers to a time period immediately after administration of the drug-containing particle wherein less than about 10%, for example less than about 9%, less than about 8%, less than about 7%, less than about 6%, less than about 5%, less than about 4%, less than about 3%, less than about 2%, less than about 1%, or more substantially about 0%, of the drug is released from a particle. In the context of in vitro dissolution testing, lag time refers to the time period immediately after exposure to dissolution conditions, wherein less than about 10%, for example less than about 9%, less than about 8%, less than about 7%, less than about 6%, less than about 5%, less than about 4%, less than about 3%, less than about 2%, less than about 1%, or more substantially about 0%, of the drug is released from the drug-containing particle.

As used herein, the term “immediate release” (IR) refers to release of greater than or equal to about 50% (especially if taste-masked for incorporation into an orally disintegrating tablet), in some embodiments greater than about 75%, in other embodiments greater than about 90%, and in still other embodiments greater than about 95% of the drug within about 2 hours, or in other embodiments within about one hour following administration of the dosage form.

As used herein, the term “immediate-release core” refers to a core as defined herein comprising a drug and an alkaline agent, optionally layered with a sealant layer, wherein the optional sealant layer functions to protect the immediate-release core from attrition and abrasion, but does not provide any substantial controlled-release properties. An “immediate-release core” can include drug crystals (or amorphous particles), an alkaline agent and granules or granulates of the drug with one or more excipients, an inert core (e.g., a sugar sphere) layered with a drug (and an optional binder), an optional protective sealant coating, and an alkaline buffer layer, or an alkaline agent layered with a drug (and an optional binder), and an optional protective sealant coating. Immediate-release cores have immediate release properties as described herein. Controlled-release particles (e.g., extended-release particles; sustained-release particles; delayed-release particles; timed, pulsatile release-particles, etc.) can be prepared by coating immediate-release cores with one or more controlled-release coatings.

As used herein, the term “sustained-release” (SR) refers to the property of slow release of a drug from a drug-containing core particle, without an appreciable lag time. The term “sustained-release coating” or “SR coating” refers to a coating showing sustained-release properties. The term “sustained release (SR) bead” or “sustained release particle” refers broadly to a bead or particle comprising an SR coating, as described herein, disposed over a drug-containing core coated with an SR coating as described herein. In one embodiment, a sustained-release coating comprises a water-insoluble polymer and optionally a water-soluble polymer. An SR coating can optionally contain a plasticizer or other ingredients that do not interfere with the “sustained-release” properties of the coating.

As used herein, the term “timed, pulsatile release” (TPR) refers to the property of modified release of a drug after a pre-determined lag time. The term “timed, pulsatile-release coating” or “TPR coating” refers to a coating showing timed, pulsatile-release properties. The term “lag-time coating” or “TPR coating” refers to a controlled-release coating comprising the combination of water-insoluble and enteric polymers as used herein. A TPR coating by itself provides an immediate release pulse of the drug, or a sustained drug-release profile after a pre-determined lag time. The term “lag-time bead,” “lag-time particle,” “TPR bead” or “TPR particle” refers broadly to a bead or particle comprising a TPR coating, as described herein, disposed over a drug-containing core.

In some embodiments, a lag time of from at least about 2 to about 10 hours is achieved by coating the particle with, e.g. a combination of at least one water-insoluble polymer and at least one enteric polymer (e.g., a combination of ethylcellulose and hypromellose phthalate). A TPR coating can optionally contain a plasticizer or other ingredients which do not interfere with the “timed, pulsatile release” properties of the coating.

As used herein, the term “delayed release” (DR) refers to the property of immediate release of a drug after a predetermined lag time. The term “delayed release coating” or “DR coating” refers to a coating showing delayed-release properties. The term “delayed release particle” refers to a drug-containing particle showing delayed-release properties. In some embodiments, a lag time of from at least about 2 to about 10 hours is achieved by coating the particle with an enteric polymer (e.g., hypromellose phthalate). A delayed-release coating can optionally contain a plasticizer or other ingredients which do not interfere with the delayed-release properties of the coating.

The term “disposed over,” e.g. in reference to a coating over a substrate, refers to the relative location of e.g. the coating in reference to the substrate, but does not require that the coating be in direct contact with the substrate. For example, a first coating “disposed over” a substrate can be in direct contact with the substrate, or one or more intervening materials or coatings can be interposed between the first coating and the substrate. In other words, for example, an SR coating disposed over a drug-containing core can refer to an SR coating deposited directly over the drug-containing core, or can refer to an SR coating deposited onto a protective seal coating deposited on the drug-containing core.

The term “sealant layer” refers to a protective membrane disposed over a drug-containing core particle.

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution”, in that “disintegration” refers to the breaking up of or loss of structural cohesion of e.g. the constituent particles comprising a tablet, whereas “dissolution” refers to the solublization of a solid in a liquid (e.g., the solublization of a drug in solvents or gastric fluids).

The term “substantially masks the taste” in reference to the taste-masking layer of IR particles in a dosage form (when present), refers to the property of the taste-masking layer of substantially preventing the release or dissolution of the drug in the oral cavity of a patient, thereby preventing the patient from tasting the drug. A taste-masking layer which “substantially masks” the taste of the drug typically releases less than about 10% of the drug in the oral cavity of the patient, in other embodiments, less than about 5%, less than about 1%, less than about 0.5%, less than about 0.1%, less than about 0.05%, less than about 0.03%, less than about 0.01% of the drug. The taste-masking properties of the taste-masking layer of the compositions of the present invention can be measured in vivo (e.g., using conventional organoleptic testing methods known in the art) or in vitro (e.g., using dissolution tests as described herein). The skilled artisan will recognize that the amount of drug release associated with a taste-masking layer that “substantially masks” the taste of a drug is not limited to the ranges expressly disclosed herein, and can vary depending on other factors such as the perceived bitterness of the drug and, e.g. the presence of flavoring agents in the composition.

The term “substantially free” means that the ingredient indicated is not present, or is present in only insignificant amounts. In one embodiment, “substantially free” means less than about 10%. In other embodiments, “substantially free” means less than about 5%, less than about 2%, or less than about 1%, or about 0%. For example, a coating that is substantially free of water-insoluble polymers does not contain any water-insoluble polymer in a substantial amount. The term “substantially free of water-insoluble polymers” does not exclude polymers that are water-soluble or water-insoluble ingredients that are not polymers. The term “water-insoluble polymer” refers to a polymer which is insoluble or very sparingly soluble in aqueous media, independent of pH, or over a broad pH range (e.g., pH 1.0 to pH 14). A polymer that swells but does not dissolve in aqueous media can be “water-insoluble,” as used herein.

The term “water-soluble polymer” refers to a polymer which is soluble (i.e., a significant amount dissolves) in aqueous media, independent of pH.

The term “enteric polymer” refers to a polymer which is soluble (i.e., a significant amount dissolves) under intestinal conditions; i.e., in aqueous media under˜neutral to alkaline conditions and insoluble under acidic conditions (i.e., low pH).

The term “reverse enteric polymer” refers to a polymer that is soluble under acidic conditions and insoluble at neutral and alkaline conditions.

The terms “plasma concentration vs. time profile,” “Cₘₐₓ,” “AUC,” “Tₘₐₓ” and “elimination half life” have their generally accepted meanings as defined in the FDA Guidance for Industry: Bioavailability and Bioequivalence Studies for Orally Administered Drug Products—General Considerations (issued March 2003).

Unless stated otherwise, the amount of the various coatings or layers described herein (the “coating weight”) is expressed as the percentage weight gain of the particles or beads provided by the dried coating, relative to the initial weight of the particles or beads prior to coating. Thus, a 10% coating weight refers to a dried coating which increases the weight of a particle by 10%.

In one embodiment, the present invention relates to a pharmaceutical composition comprising a first population of controlled-release particles, wherein the controlled-release particles of the first population comprises a core comprising a proton pump inhibitor or a pharmaceutically acceptable salt, solvate, and/or ester thereof, and an alkaline agent; a first coating disposed over said core, comprising an enteric polymer; and a second coating disposed over the core, comprising an enteric polymer and a water-insoluble polymer, wherein the first coating is substantially free of water-insoluble polymers.

In one embodiment, the drug-containing core can take the form of a bead, a pellet, a granulate, a microgranule, a drug crystal, a mini-tablet, etc. In another embodiment, the core comprises an inert bead coated with a layer containing the drug.

The proton pump inhibitors (PPIs) of the present invention encompass drugs that reduce gastric acid production by inhibiting the hydrogen/potassium adenosine triphosphatase enzyme system of the gastric parietal cell. The PPIs of the present invention do not encompass H₂-histamine receptor antagonists or anticholinergic agents. A non-limiting list of PPIs suitable for use in the compositions of the present invention include pantoprazole, rabeprazole, pariprazole, lemiprazole, omeprazole, esomeprazole, lansoprazole, tenatoprazole, nepaprazole, ilaparazole, etc. or a pharmaceutically acceptable salt, solvate, hydrate, polymorph and/or ester thereof, and mixtures thereof. In one embodiment, the PPI is pantoprazole sodium, or a hydrate of pantoprazole sodium (e.g., a sesquihydrate).

Pantoprazole is the active ingredient (as the sodium salt) in the marketed product, Protonix®. Pantoprazole can be abbreviated as “PTP.” Pantoprazole is rapidly absorbed in the intestine with a mean Tₘₐₓ of approximately 90 minutes after oral administration. It is eliminated quickly with a short half-life (t_{1/2}) of 1.8 hours. The Cₘₐₓ and AUC are dose proportional over 10 mg to 80 mg doses. Pantoprazole does not accumulate upon multiple dosing. Food delays absorption without altering Cₘₐₓ and AUC. It is taken, like any other PPI, 1 hour to 30 minutes before meals. Pantoprazole is chemically 5-(difluoromethoxy)-2-[(3,4-dimethoxypyridin-2-yl) methylsulfinyl]-3H-benzoimidazole, with an empirical formula of C₁₆H₁₅F₂N₃O₄S and a molecular weight of 383.37. Its chemical structure is shown below. Pantoprazole sodium sesquihydrate is a white to off-white crystalline powder, with weakly basic properties. Pantoprazole sodium sesquihydrate is freely soluble in water, but very slightly soluble in phosphate buffer at pH 7.4.

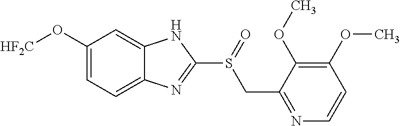

In one embodiment of the pharmaceutical compositions of the present invention, the first coating is disposed over the core, and the second coating is disposed over the first coating. In another embodiment, the second coating is disposed over the core and the first coating is disposed over the second coating.

In one embodiment of the pharmaceutical compositions of the present invention, at least one sealant layer disposed over the core; for example, the sealant layer underlies the first and second coatings. The sealant layer comprises a hydrophilic polymer. Non-limiting examples of polymers suitable for use in the sealant layer include hydroxypropylcellulose (e.g., Klucel® LF), hydroxypropyl methylcellulose or hypromellose (e.g., Opadry® Clear or Pharmacoat™ 603), vinylpyrrolidone-vinylacetate copolymer (e.g., Kollidon® VA 64 from BASF), and low-viscosity ethylcellulose (e.g., viscosity of 10 cps or less, optionally in combination with hydroxypropylcellulose). The sealant layer can constitute from about 1% to about 10% of the weight of the controlled-release particle, for example about 1%, about 2%, about 3%, about 4%, about 5%, about 7%, about 8%, about 9%, or about 10%, inclusive of all ranges and subranges therebetween.

It is well-known in the art that a polymer can be both water insoluble and hydrophilic. For example, certain cellulose derivatives may fall under these categories, depending on degree of substitution, viscosity, processing parameters, etc. To illustrate, certain grades of ethylcellulose would be considered water insoluble (e.g., viscosity of about 90-110 cps), while other grades of ethylcellulose would be considered hydrophilic (e.g., low viscosity, or viscosity about 10 cps or lower) although they are water insoluble.

In one embodiment, the pharmaceutical compositions of the present invention further comprise a compressible coating disposed over the first and second coatings. In one embodiment, the compressible coating comprises at least one hydrophilic polymer. Suitable hydrophilic polymers include, for example hydroxypropylcellulose, poly(vinyl acetate-vinyl pyrrolidone), polyvinyl acetate, ethylcellulose, and mixtures thereof. In another embodiment, the compressible coating comprises hydroxypropylcellulose. In another embodiment, the compressible coating comprises a water-insoluble polymer and optionally a plasticizer (e.g., ethylcellulose and an optional plasticizer, such as diethyl phthalate). In still another embodiment, the compressible coating comprises from about 2% to about 10% by weight of the controlled-release particle.

In another embodiment, the pharmaceutical compositions of the present invention comprise a sealant layer underlying the controlled-release layers and a compressible coating disposed over the controlled-release layers. In another embodiment, the sealant layer and the compressible coating each comprise a hydrophilic polymer. In another embodiment, the sealant layer and the compressible coating each independently comprise a polymer selected from the group consisting of hydroxypropylcellulose, hydroxypropyl methylcellulose, poly(vinyl acetate-vinyl pyrrolidone), polyvinyl acetate, low-viscosity ethylcellulose, and mixtures thereof.

In some embodiments, the pharmaceutical compositions of the present invention comprise controlled-release particles, comprising a core, first coating disposed over the core (wherein the first coating comprises an enteric polymer); and a second coating disposed over the core comprising the combination of an enteric polymer and a water-insoluble polymer. In one embodiment, the first coating is substantially free of water-insoluble polymers.

Non-limiting examples of suitable enteric polymers include anionic polymers. Further non-limiting examples of enteric polymers include hydroxypropyl methylcellulose phthalate, cellulose acetate phthalate, hydroxypropyl methylcellulose acetate succinate, polyvinyl acetate phthalate, pH-sensitive methacrylic acid-methylmethacrylate copolymers, shellac, and mixtures thereof. These enteric polymers may be used as a dry powder or an aqueous dispersion. Some commercially available materials that may be used are methacrylic acid copolymers sold under the trademark Eudragit® (L100, S100, L30D, FS30D) manufactured by Rohm Pharma, Cellacefate® (cellulose acetate phthalate) from Eastman Chemical Co., Aquateric® (cellulose acetate phthalate aqueous dispersion) from FMC Corp., and Aqoat® (hydroxypropyl methylcellulose acetate succinate aqueous dispersion) from Shin Etsu K.K.

Examples of water-soluble polymers include (but are not limited to) methylcellulose, hydroxypropyl cellulose, hydroxypropyl methylcellulose, polyethylene glycol, and polyvinyl pyrrolidone.

In one embodiment, the coating weight of the first coating ranges from about 10% to about 60% of the total weight of the controlled-release particle, including from about 10% to about 55%, about 10% to about 50%, about 10% to about 45%, about 10% to about 40%, and all subranges therebetween. In another embodiment, the coating weight of the second coating ranges from about 5% to about 60%, about 10% to about 60%, about 15% to about 60%, about 20% to about 60%, about 5% to about 55%, about 5% to about 50%, about 5% to about 45%, about 5% to about 40%, about 5% to about 35%, about 15% to about 55%, about 20% to about 50%, or about 25% to about 45%, or about 10% to about 40%, of the total weight of the controlled-release particle. In another embodiment, the weight of the first and second coatings in combination range from about 20% to about 70% by weight of the total weight of the controlled-release particle. In one embodiment, the second controlled-release coating is disposed over the first controlled-release coating, which, in turn, is disposed over the core. In this embodiment, the weight of the first coating ranges from about 10% to about 60% of the total weight of the singly-coated controlled-release particle before the second coating is applied, and, once applied, the weight of the second coating ranges from about 5% to about 60%, or about 10% to about 40% of the total weight of the dual-coated controlled-release particle.

In another embodiment, the first controlled-release coating is disposed over the >0 second controlled-release coating. In this embodiment, the second coating ranges from about 5% to about 60%, or about 15% to about 60% of the total weight of the singly-coated controlled-release particle before the first coating is applied, and, once applied, the first coating ranges from about 10% to about 40% of the total weight of the dual-coated controlled-release particle.

In one embodiment, the enteric polymer of the first coating comprises hydroxypropyl methylcellulose phthalate. In another embodiment, the enteric polymer of the second coating comprises hydroxypropyl methylcellulose phthalate. In another embodiment, the first and second coatings both comprise the same enteric polymer (e.g., hydroxypropyl methylcellulose phthalate).

Non-limiting examples of water-insoluble polymers include ethylcellulose, cellulose acetate, cellulose acetate butyrate, polyvinyl acetate, neutral methacrylic acid-methylmethacrylate copolymers, and mixtures thereof. In one embodiment, the water-insoluble polymer is ethylcellulose. In another embodiment, the water-insoluble polymer comprises ethylcellulose with a mean viscosity of 10 cps in a 5% solution in 80/20 toluene/alcohol measured at 25° C. on an Ubbelohde viscometer.

In certain embodiments of the present invention, the second controlled-release coating comprises an enteric polymer and a water-insoluble polymer. In one embodiment, the weight ratio of the water-insoluble polymer to the enteric polymer in the second coating ranges from about 10:1 to about 1:4, including the ranges of from about 9:1 to about 1:3 and from about 3:1 to about 1:1, and all subranges therebetween. In another embodiment, the enteric and water-insoluble polymers in the second coating in combination constitute from about 5% to about 60% by weight of the total weight of the controlled-release particle, including the ranges of from about 10% to about 60%, from about 10% to about 40%, and all subranges therebetween.

Any of the controlled-release coatings of the controlled-release particles can independently further comprise a plasticizer. For example, the first controlled-release coating, or the second controlled-release coating, or both can comprise a plasticizer. Non-limiting examples of suitable plasticizers include glycerol and esters thereof (e.g., monoacetylated glycerides, acetylated mono- or diglycerides (e.g., Myvacet® 9-45)), glyceryl monostearate, glyceryl triacetate, glyceryl tributyrate, phthalates (e.g., dibutyl phthalate, diethyl phthalate, dimethylphthalate, dioctylphthalate), citrates (e.g., acetylcitric acid tributyl ester, acetylcitric acid triethyl ester, tributyl citrate, acetyltributyl citrate, triethyl citrate), glyceroltributyrate; sebacates (e.g., diethyl sebacate, dibutyl sebacate), adipates, azelates, benzoates, chlorobutanol, polyethylene glycols, vegetable oils, fumarates, (e.g., diethyl fumarate), malates, (e.g., diethyl malate), oxalates (e.g., diethyl oxalate), succinates (e.g., dibutyl succinate), butyrates, cetyl alcohol esters, malonates (e.g., diethyl malonate), castor oil, and mixtures thereof. When used in an embodiment of the present invention, the plasticizer may constitute from about 3% to about 30% by weight of the polymer(s) in the controlled-release coating. In still other embodiments, the amount of plasticizer relative to the weight of the polymer(s) in the controlled-release coating is about 3%, about 5%, about 7%, about 10%, about 12%, about 15%, about 17%, about 20%, about 22%, about 25%, about 27%, and about 30%, inclusive of all ranges and subranges therebetween. One of ordinary skill in the art will recognize that the presence of plasticizer, or type(s) and amount(s) of plasticizer(s) can be selected based on the polymer or polymers and nature of the coating system (e.g., aqueous or solvent-based, solution or dispersion-based and the total solids). For example, the amount of plasticizer required depends upon the plasticizer, the properties of the water-insoluble polymer, and the ultimate desired properties of the coating.

In most embodiments of the present invention, the core comprises an alkaline agent (in addition to a proton pump inhibitor). Non-limiting examples of suitable alkaline agents include sodium carbonate, sodium bicarbonate, sodium hydroxide, monosodium dihydrogen phosphate, disodium hydrogen phosphate, trisodium phosphate, sodium acetate, sodium silicate, magnesium carbonate, magnesium oxide, magnesium hydroxide, magnesium metasilicate aluminate, magnesium silicate aluminate, magnesium silicate, aluminum magnesium hydroxide, magnesium phosphate, magnesium acetate, magnesium carbonate, complex magnesium aluminum metasilicate, calcium carbonate, calcium hydroxide, potassium carbonate, calcium silicate, monopotassium dihydrogen phosphate, dipotassium hydrogen phosphate, tripotassium phosphate, potassium acetate, and mixtures thereof. In one embodiment, the ratio of the proton pump inhibitor to the alkaline agent in the core ranges from about 7:1 to about 1:3, about 6:1 to about 1:2, about 5:1 to about 1:1, and about 4:1 to about 1:1. In other embodiments, the ratio of the proton pump inhibitor to the alkaline buffer ranges from about 5:1 to about 1:5, about 4:1 to about 1:4, about 3:1 to about 1:3, and about 2:1 to about 1:2, inclusive of all subranges therebetween.

The alkaline agent may be present in any location in the controlled-release particle. For example, the alkaline agent may be in contact with the drug or may be located separately. In certain embodiments, the core of the controlled-release particle may take the form of an inert bead coated with a drug layer, and the alkaline agent may be present in the inert bead, or in some embodiments the inert bead itself may be an alkaline agent. In other embodiments, the alkaline agent may be in contact with the proton pump inhibitor. For example, the drug layer coating the inert bead may include both the drug and alkaline agent, and optionally a binder. In another embodiment, the alkaline agent may be present in a coating separate from the drug layer; for example, in a separate layer overlying or underlying the drug layer. The alkaline agent may be a base (e.g., an alkali, alkaline earth, or other metal hydroxide) or a buffer (e.g., the alkali or alkaline earth or other metal salt of a weak base). In some embodiments, the alkaline agent is not in contact with the drug. In some embodiments the alkaline buffer layer is disposed on a sealant layer, which in turn is disposed on a core comprising a proton pump inhibitor.

In one embodiment of the present invention, the core comprises an inert bead coated with a buffer layer comprising an alkaline buffer as the alkaline agent, disposed over the inert bead and underlying the first and second coatings. The alkaline buffer layer is believed to create an alkaline microenvironment at the drug interface inside the controlled-release particle. Because the proton pump inhibitor has a lower solubility in this microenvironment, the alkaline buffer layer effectively delays release of the drug under the acidic to neutral pH conditions of the gastrointestinal tract, conditions under which the drug would otherwise dissolve rapidly. By incorporating an alkaline buffer layer into the compositions of the present invention, it is possible to achieve pharmacokinetic profiles suitable for a once- or twice-daily dosing regimen. However, compositions of the present invention are not limited to those which function by this mechanism.

In embodiments of the compositions of the present invention in which the alkaline agent is present in a separate layer, the alkaline agent-containing layer optionally further comprises a polymeric binder. The polymeric binder can be any of those disclosed herein, for example hydroxypropylcellulose, polyvinylpyrrolidone (povidone), methylcellulose, hydroxypropyl methylcellulose, carboxyalkylcellulose, polyethylene oxide, starch, polysaccharides, etc.

The core of the controlled-release particle comprises a proton pump inhibitor (in addition to the alkaline agent). In some embodiments, the core can take the form of a drug granule or granulate (e.g., comprising particles of the drug granulated in the present of pharmaceutically acceptable excipients), a drug crystal, or an inert bead coated with a drug layer comprising a proton pump inhibitor or a pharmaceutically acceptable salt, ester, and/or solvate thereof. When in the form of an inert bead, the core comprises, for example, sugar, microcrystalline cellulose, lactose, mannitol-microcrystalline cellulose, lactose-microcrystalline cellulose, silicon dioxide, etc. In one embodiment, the core has an average particle size of not more than about 400 μm, or, in another embodiment, not more than about 350 μm. In one embodiment, the drug layer comprises a polymeric binder, as described herein. The ratio of the proton pump inhibitor to the polymeric binder can range from about 85:15 to about 100:0 (no binder). In most embodiments, the drug layer also comprises an alkaline agent, as described herein.

In one embodiment of the present invention, a first population of controlled-release particles exhibit a drug release profile substantially corresponding to the following pattern when dissolution tested using United States Pharmacopoeia Apparatus 2 (paddles @ 50 rpm) in a 2-stage dissolution media (700 mL of 0.1N HCl for the first 2 hours followed by testing in 900 mL buffer at pH 6.8 obtained by adding 200 mL of a pH modifier) at 37° C.:
after 1 hour, no more than about 30% of the total amount of proton pump inhibitor is released;
after 4 hours, from about 30-70% of the total amount of proton pump inhibitor is released; and
after 12 hours, not less than about 60% of the total amount of proton pump inhibitor is released.

In another embodiment, a first population of controlled-release particles provides a lag time of from about 1 hour to about 6 hours, followed by release of the proton pump inhibitor over a period of from about 2 hours to about 6 hours. In another embodiment, a first population of controlled-release beads provides a lag time of from about 1 hour to about 4 hours, followed by release of the proton pump inhibitor over a period of from about 4 hours to about 8 hours.

The drug release profiles of the controlled-release particles may be determined by dissolution testing in a USP Apparatus 1 or 2 using a two-stage dissolution medium (first 2 hours in 700 mL of 0.1N HCl at 37° C. followed by dissolution testing at pH 6.8 obtained by the addition of 200 mL of a pH modifier). Drug release over time can be determined using various methods; for example, quantification of drug in solution (e.g., as measured by ultraviolet absorption) on samples pulled at selected time points during dissolution testing and subjected to high performance liquid chromatography (HPLC). In the dissolution media at pH 6.8, the pantoprazole hydrochloride undergoes slow reduction to the base which is not very soluble, and the analytical method is capable of quantifying and correcting for the change in potency.

The controlled-release coating(s) contributes to the control of drug dissolution at the drug interface and hence drug release from the controlled-release particles. The achievable lag time or sustained-release time depends on the composition and thickness of the controlled-release coating(s). Some factors that can affect drug dissolution include, but are not limited to, the pKa of the drug, the solubility of the drug, the elimination half-life of the drug, solubility reduction in the micro-alkaline pH environment created by the alkaline agent (if present), and the alkaline agent used (if present).

In certain embodiments, the pharmaceutical composition described above further comprises a second population of controlled-release particles. The particles of this second population comprise a core comprising a proton pump inhibitor or a pharmaceutically acceptable salt, solvate, and/or ester thereof; and at least one controlled-release coating disposed over the core, comprising an enteric polymer.

The second population of controlled-release particles may have characteristics similar to those of the first population of controlled-release particles. The core in the second population of controlled-release particles comprises a drug granule or granulate, a drug crystal, a mini-tablet, a pellet, or an inert bead coated with a drug layer comprising said proton pump inhibitor or a pharmaceutically acceptable salt, ester, and/or solvate thereof. Suitable proton pump inhibitors include those described herein for the first population of controlled-release particles. Suitable enteric polymers include those described herein for the first population of controlled-release particles. The second population of controlled-release particles may further comprise a sealant layer (for example, underlying the controlled-release coating(s)) and/or a compressible coating (for example, disposed over the controlled-release coating(s)). If present, the sealant layer and/or compressible coating may independently comprise a hydrophilic polymer, as described herein. In one embodiment, the second population of controlled-release particles comprise a plasticizer and/or an alkaline agent, as described herein.

In one embodiment of the present invention, the weight of the controlled-release coating of the second population of controlled-release particles ranges from about 10% to about 60% of the total weight of the coated controlled-release particles, including about 15%, about 20%, about 25%, about 30%, about 35%, about 40%, about 45%, about 50%, or about 55%, inclusive of all values and ranges therebetween.

In one embodiment, the controlled-release particles in said second population release at least about 75% of said proton pump inhibitor within about 60 minutes when tested for dissolution in USP Apparatus 1 (baskets at 100 rpm) or Apparatus 2 (paddles at 50 rpm) in 900 mL buffer at pH 6.8 at 37° C. In another embodiment, the controlled-release particles in the second population release at least about 80%, at least about 85%, at least about 90%, at least about 95%, or provide substantially complete release of the proton pump inhibitor within about 60 minutes when tested as described herein.

In most embodiments, the pharmaceutical compositions of the present invention exhibit a bimodal pulsatile release profile providing two peaks in blood plasma concentration of a proton pump inhibitor, separated by about 1 to about 6 hours. In another embodiment, the first and second populations of controlled-release particles start releasing the proton pump inhibitor at a substantially different rates (e.g. the release rates of the first and second populations of controlled-release particles differ by at least about 10%, or at least about 20%, or least about 30%, or least about 40%, or least about 50%, or least about 60%, or least about 70%, or least that 80%, release that 90%, or least 100%). In yet another embodiment, the first population of controlled-release particles exhibits a lag time of about 1 to about 6 hours, followed by release of the proton pump inhibitor contained therein over a period of about 2 hours to about 6 hours, or about 4 hours to about 8 hours; and the second population of controlled-release particles provides substantially complete release of said proton pump inhibitor contained therein upon entry into the intestine, or after exposure to the dissolution conditions described herein. In one embodiment, the first population of controlled-release particles releases drug from about 1 hour to about 6 hours after the second population of controlled-release particles releases drug. In another embodiment, the first population of controlled-release particles provides a peak in drug plasma levels from about 1 hour to about 6 hours after the peak provided by the second population of controlled-release particles. In another embodiment, the Cₘₐₓ of the blood plasma peak provided by the first controlled-release population occurs from about 1 hour to about 6 hours later than the Cₘₐₓ of the blood plasma peak provided by the second controlled-release population.

In some embodiments, the ratio of the second population of controlled-release particles to the first population of controlled-release particles ranges from about 25:75 to about 75:25, including about 25:75, about 30:70, about 35:65, about 40:60, about 45:55, about 50:50, about 55:45, about 60:40, about 65:35, about 70:30, or about 75:25, inclusive of all ranges and subranges therebetween.

The pharmaceutical compositions described herein (comprising a single population of controlled-release particles or a combination of a first population and a second population of controlled-release particles) can further comprise rapidly disintegrating granules, wherein the rapidly disintegrating granules comprise a saccharide and/or a sugar alcohol in combination with a disintegrant. The disintegrant can be selected from the group consisting of crospovidone, sodium starch glycolate, crosslinked sodium carboxymethylcellulose, and low-substituted hydroxypropylcellulose. Suitable saccharides and sugar alcohols include lactose, sucralose, sucrose, maltose, mannitol, sorbitol, xylitol, glycol, glycerol, erythritol, arabitol, ribitol, isomalt, lactitol, and maltitol. The ratio of the disintegrant to the saccharide and/or sugar alcohol in the rapidly dispersing microgranules ranges from about 1:99 to about 10/:90 (including about 1:99, about 2:98, about 3:97, about 4:96, about 5:95, about 6:94, about 7:93, about 8:92, about 9:91, or about 10:90, inclusive of all ranges therebetween), and in other embodiments is about 5/95 (by weight). In some embodiments, the disintegrant or the saccharide and/or sugar alcohol, or both, can be present in the form of microparticles having an average particle size of about 30 μm or less. The ratio of the drug-containing microparticles to the rapidly disintegrating granules can range from about 1:6 to about 1:2, including about 1:6, about 1:5, about 1:4, about 1:3, or about 1:2, inclusive of all ranges therebetween.

The present invention relates to pharmaceutical compositions and dosage forms comprising the controlled-release particles described herein. The pharmaceutical dosage forms include orally disintegrating tablets (ODTs), tablets, and capsules. When the pharmaceutical dosage form is a conventional rapidly dispersing tablet, the conventional tablet comprises microparticles of the present invention, combined as needed with any pharmaceutically acceptable excipient(s), such as binders (e.g., polymeric finders), disintegrants, fillers, diluents, Ludiplus® (lactose/poly(vinyl acetate-vinyl pyrrolidone)), Prosolv® (microcrystalline cellulose/fused silicon dioxide), lubricants, etc. When the pharmaceutical dosage form is a capsule, a capsule is filled with at least one population of controlled-release particles of the present invention. The capsule can be a gelatin capsule, a polysaccharide capsule, a HPMCP capsule, etc. When the pharmaceutical dosage form takes the form of an ODT (e.g., comprising at least one population of controlled-release particles as described herein and rapidly disintegrating granules as described herein), the ODT substantially disintegrates within about 60 seconds after contact with saliva in the oral cavity or with simulated saliva fluid. In another embodiment, the ODT substantially disintegrates within about 30 seconds after contact with saliva in the oral cavity or with simulated saliva fluid. Disintegration may be tested, for example, according to USP <701> Disintegration Test. In most embodiments, the ODT comprises a therapeutically effective amount of a proton pump inhibitor, wherein after administration the ODT substantially disintegrates in the oral cavity of a patient forming a smooth, easy-to-swallow suspension having no gritty mouthfeel or aftertaste. In most embodiments, the ODT provides a target pharmacokinetic profile (i.e., plasma concentration vs. time plot) of the proton pump inhibitor suitable for a once- or twice-daily dosing regimen.

In one embodiment of the pharmaceutical dosage form described herein, the dosage form comprises a tablet having a friability of less than about 1%. In another embodiment, the tablet has a mean hardness value of from about 20 N to about 80 N. When the pharmaceutical dosage form is an ODT, the tablet may also include pharmaceutically acceptable excipients suitable for use in disintegrating tablet formulations such as compressible diluents, fillers, coloring agents, and optionally a lubricant.

The present invention also relates to methods of preparing a pharmaceutical composition comprising at least one population of controlled-release particles as described herein. In one embodiment, the method comprises: (a) preparing a core comprising a proton pump inhibitor; (b) applying a first coating over the core, wherein the first coating comprises an enteric polymer; and (c) applying a second coating over the core, wherein the second coating comprises an enteric polymer and a water-insoluble polymer. In one embodiment, the first coating is applied before the second coating is applied. In another embodiment, the second coating is applied before the first coating. The step of preparing the core may be accomplished by any of the methods known in the art; for example, layering an inert bead (e.g., sugar, microcrystalline cellulose, mannitol-microcrystalline cellulose, silicon dioxide, etc.) with a solution comprising the drug and optionally a polymeric binder (e.g., by fluid-bed or pan coating); granulating the drug with an appropriate diluent (e.g., microcrystalline cellulose and/or lactose and optionally an alkaline agent); extruding and spheronizing the drug mixture (e.g. combined with a suitable excipients and optionally an alkaline agent); compressing the drug (and optionally excipients and alkaline agents) into mini-tablets of about 100 μm to about 10 mm (e.g., about 0.2-2 mm) in diameter; or simply obtaining drug crystals of the desired particle size (e.g., about 50-500 μm, including 100-400 μm). When the core takes the form of an inert bead, the solution of drug-layering solution applied to the inert bead may comprise an alkaline agent. In another embodiment, the step of preparing the core further comprises applying a separate layer comprising an alkaline agent. In alternative embodiments, the inert bead itself may comprise an alkaline agent.

The present invention further relates to methods of preparing pharmaceutical dosage forms comprising at least one population of extended-release particles as described herein (e.g., tablets, capsules, or orally disintegrating tablets). In one embodiment, the method comprises: mixing controlled-release particles with rapidly dispersing granules comprising a saccharide and/or sugar alcohol in combination with a disintegrant, thereby forming a compressible blend; and compressing the compressible blend into an orally disintegrating tablet. In another embodiment, the step of mixing controlled-release particles comprises mixing first and second populations of controlled-release particles with the rapidly dispersing granules, wherein the first and second populations of controlled-release particles exhibiting different drug release profiles. In another embodiment, the method comprises filling controlled-release particles as described herein into a capsule. In another embodiment, the method comprises filling multiple populations (e.g., two) of controlled-release particles as described herein into a capsule, wherein the populations of controlled-release particles may exhibit different drug-release profiles. In still other embodiments, the method comprises combining one or more populations of controlled-release particles, optionally with additional pharmaceutically acceptable excipients, and compressing this combination into a conventional tablet.

The present invention still further relates to methods of administering the pharmaceutical compositions or dosage forms described herein. In one embodiment, the method comprises administering a first population of controlled-release particles, comprising: a core comprising a proton pump inhibitor or a pharmaceutically acceptable salt, solvate, and/or ester thereof, and an alkaline agent; a first coating disposed over the core, comprising an enteric polymer; and a second coating disposed over the core, comprising an enteric polymer and a water-insoluble polymer. In another embodiment, the method comprises administering a composition comprising multiple populations (e.g., two) of controlled-release particles into a capsule, wherein the populations of controlled-release particles may exhibit substantially different drug-release profiles.

In another embodiment, the present invention relates to methods of administering the pharmaceutical dosage forms comprising the compositions described herein. In one embodiment, the method comprises administering a pharmaceutical dosage form (e.g., a tablet, an orally disintegrating tablet, or a capsule) comprising one or more of the populations of controlled-release particles described herein. In one embodiment, the method comprises administering an orally disintegrating tablet comprising two populations of controlled-release particles exhibiting substantially different drug-release profiles.

The following non-limiting examples illustrate the various embodiments of the pharmaceutical compositions and dosage forms of the present invention. Such compositions and dosage forms, when properly administered, provide therapeutically effective drug plasma concentrations while minimizing the occurrence of side-effects associated with Cₘₐₓ or Cₘᵢₙ.

### EXAMPLES

### Example 1

### 1.A Pantoprazole Sodium IR Beads at a drug load of 20%

Klucel® LF (120 g Hydroxypropylcellulose) was slowly added to ethanol 96% (2625 g) until dissolved under constant stirring for not less than 10 min. Pantoprazole sodium (880 g) was then added to the polymer binder solution until dissolved. Then micronized magnesium oxide (293 g) was homogeneously suspended in the Klucel®/ethanol solution. A Glatt GPCG 3 equipped with a 7″ bottom spray Wurster 8″ high column, partition column gap of 15 mm from the ‘B’ bottom air distribution plate covered with a 200 mesh product retention screen (1.2 mm port nozzle) was charged with 2832 g of Cellets 200 (200-355 μm microcrystalline cellulose spheres from Glatt) which were predried to reduce the moisture content to 1% and sprayed with the pantoprazole solution (33% solids) at an initial rate of 16-18 g/min at an inlet air volume of 80-125 m³/hr, air atomization pressure of 1.8 bar while maintaining the product temperature of 30-35° C. The final yield was about 99% with an actual assay of 20.8% (theoretical: 21.3% by weight).

The resulting drug-layered beads (3.8 kg) were then coated with of Klucel® LF by spraying a Klucel®/ethanol solution under conditions similar to those used for drug layering. The coated beads were then dried in the same Glatt unit for 30 min to drive off residual solvents (including moisture). The resulting pantoprazole IR beads were sieved through 35 and 80 mesh screens to discard oversized particles and fines.

### 1.B Pantoprazole TPR Beads (EC-10/HP-55/TEC at 45/40/15)

Ethylcellulose (EC-10, Ethocel Premium 10 from Dow Chemicals; 210.8 g) was slowly added to a 90/10 mixture of acetone and water while stirring constantly until dissolved. Hypromellose phthalate (HP-55 from Shin Etsu Japan; 186 g) was slowly added to the EC-10 solution until dissolved, followed by the addition of triethyl citrate (TEC; 68.2 g), until the triethyl citrate was dissolved. A Glatt GPCG 3 equipped with a 6″ bottom spray Wurster 6″ insert, ‘B” bottom air distribution plate covered with a 200 mesh product retention screen, 1.2 mm port nozzle, was charged with 1070 g of IR beads from Ex. 1.A, above. The IR beads were sprayed with the ethylcellulose/hypromellose phthalate coating formulation (7.5% solids) at a product temperature of 35-36° C., atomization air pressure of 1.5 bar, inlet air flow of 70-110 m³/hr, and a spray rate of 9-12 g/min for a TPR coating level of 30% by weight. Following spraying, the coated beads were dried in the Glatt unit for 30 min to drive off residual solvents (including moisture). The resulting TPR beads were sieved to provide particles with diameters of less than 420 μm.

### 1.C. Pantoprazole TPR Beads (EC-10/HP-55/TEC at 60/25/15)

The TPR coating formulation was prepared by first dissolving EC-10 in the 90/10 acetone/water mixture, followed by HP-55 and TEC. The IR beads of Example 1.A were sprayed with the ethylcellulose/hypromellose phthalate coating formulation (7.5% solids) in the Glatt 3 as described in Example 1.B, above. Following spraying, the coated beads were dried in the Glatt unit for 30 min to drive off residual solvents (including moisture). The resulting TPR beads were sieved to provide particles with diameters of less than 420 μm.

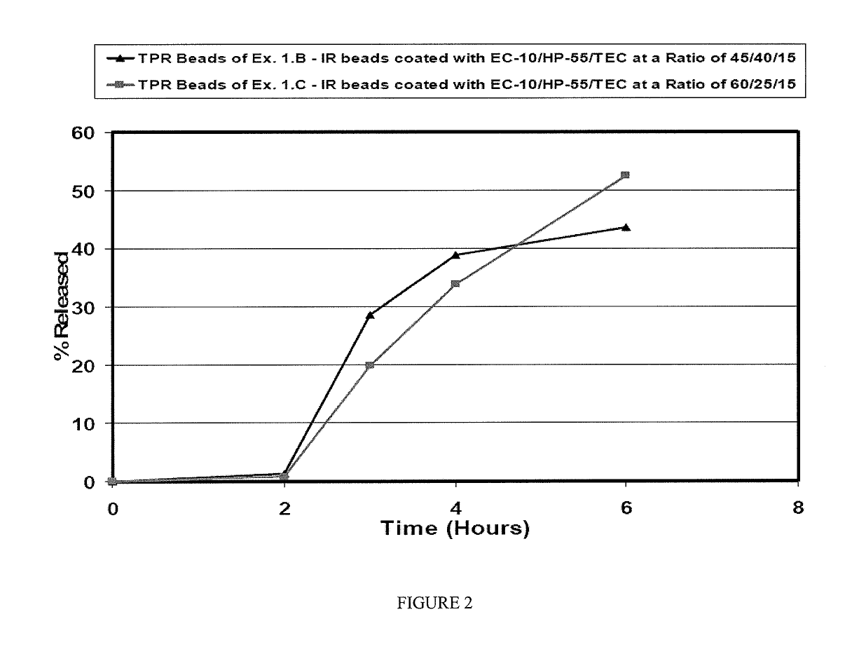

FIG. 2 demonstrates the drug release profiles for TPR beads coated at a ratio of 45/40/15 (Example 1.B) vs. 60/25/15 (Example 1.C) for a weight gain of 30%.

### Example 2

### 2.A Pantoprazole CR Beads (DR Coating on TPR (45/40/15 EC-10/HP-55/TEC) Coating)

Hypromellose phthalate (HP-55, 385.2 g) was slowly added to a 70/30 mixture of acetone and water while stirring constantly until dissolved followed by the addition of triethyl citrate (TEC; 42.8 g), until the triethyl citrate was dissolved. The TPR beads at 30% coating (1000 g) from Example 1.B, above, were fluid-bed (Glatt 3 with 6″ Wurster insert (15 mm gap)) coated with the hypromellose phthalate solution (6% solids) at a product temperature of 35±1° C., atomization air pressure of 1.5 bar, inlet air flow of 70-110 m³/hr, and a spray flow rate of 9-12 g/min for a DR coating level of 30% by weight. The resulting CR beads were dried in the Glatt unit for 30 min to drive off residual solvents. About 85% by weight of the coated beads had a size smaller than 500 μm.

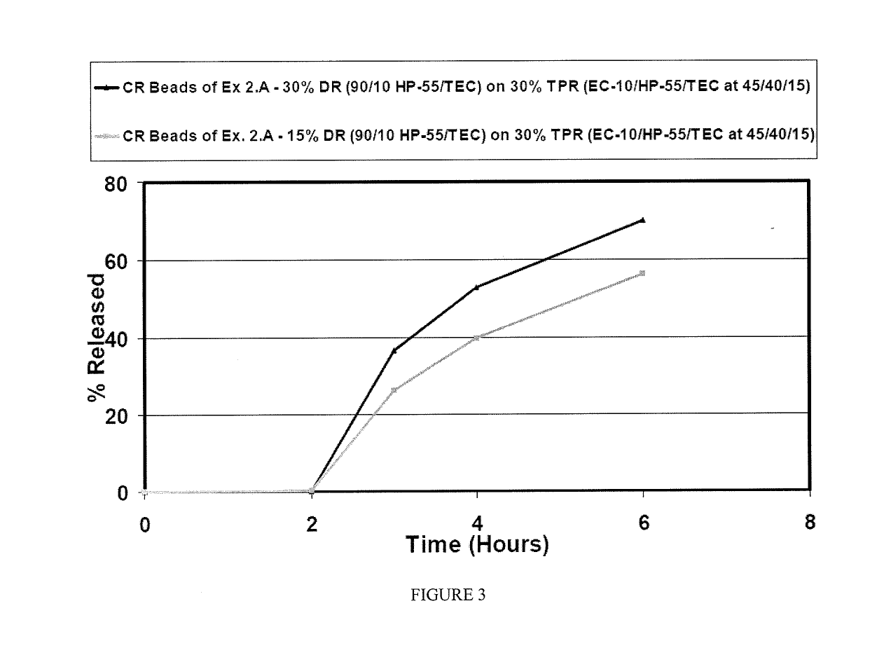

FIG. 3 shows the drug release profiles from CR beads of Example 2.A at 15% w/w or 30% w/w DR coating disposed over 30% TPR coating (45/40/15 EC-10/HP-55/TEC).

### 2.B Pantoprazole CR Beads (DR Coating on TPR (60/25/15) Coating)

Another batch of CR beads was similarly prepared (as in Example 2.A) using the TPR beads at 30% coating (1000 g TPR beads (coated with EC-10/HP-55/TEC at 60/25/15) from Example 1.C, above) by spraying a DR functional polymer solution containing HP-55/TEC 90/10 (at 6% solids) for a weight gain of 30% by weight. A sample was pulled at 15% coating for analytical testing (i.e., HPLC assay and drug release).

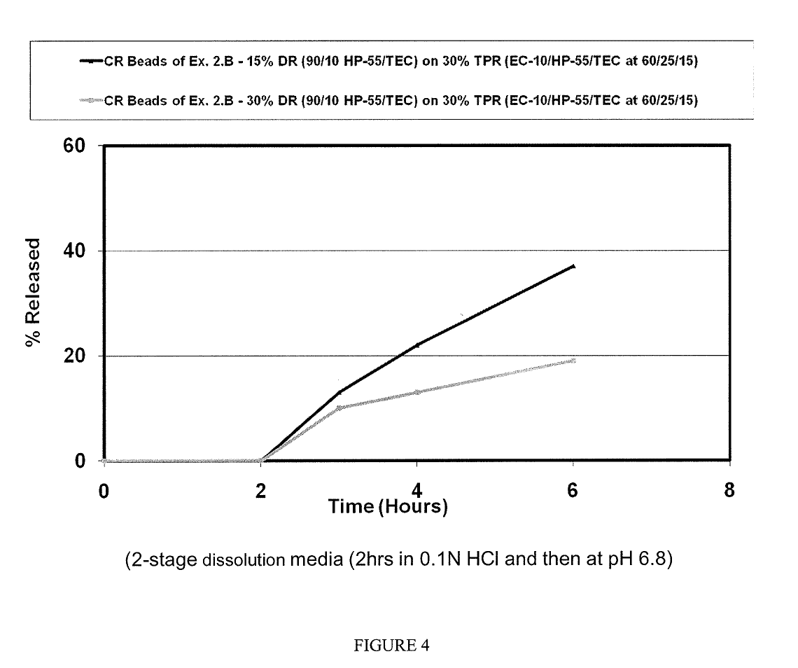

FIG. 4 shows the drug release profiles from CR beads of Example 2.B at 15% w/w versus 30% w/w DR coating (HP-55/TEC) disposed over 30% TPR coating (60/25/15 EC-10/HP-55/TEC). The drug release at 30% coating is significantly slower than at 15% coating.

### Example 3

### 3.A Pantoprazole DR Beads (HP-55/TEC at 90/10)

Hypromellose phthalate (HP-55; 229.5 g) was slowly added to a 70/30 mixture of acetone and water while stirring rigorously until dissolved. TEC (25.5 g) was added to the solution until dissolved/dispersed homogeneously. The IR beads (1000 g) from Example 1.A were fluid-bed coated with the hypromellose phthalate coating solution (6% solids) in the Glatt 3 equipped with the 6″ Wurster insert at a product temperature of 35±1° C., atomization air pressure of 1.5 bar, inlet air volume of 70-110 m³/hr, and an initial flow rate of 9-12 g/min for a DR-coating level of 50% by weight. The samples pulled at a coating of 20% was also subjected to analytical testing (i.e., HPLC assay and drug release).

### 3.B Pantoprazole CR Beads (TPR-Coating (EC-10/HP-55/TEC at 60/25/15 on DR-Coating)

The IR beads (1000 g) from Example 1.A were coated with the DR coating formulation (hypromellose phthalate/triethyl citrate at a ratio of 90/10 at 6% solids) in the Glatt 3 for a DR-coating level of 20% by weight. The resulting DR beads were then coated with a TPR coating (EC-10/HP-55/TEC at 60/25/15) for a weight gain of 30%. A sample was pulled at 15% coating and subjected to analytical testing (i.e., HPLC assay and drug release). The resulting controlled-release beads at 30% coating with particles<500 μm were collected by sieving.

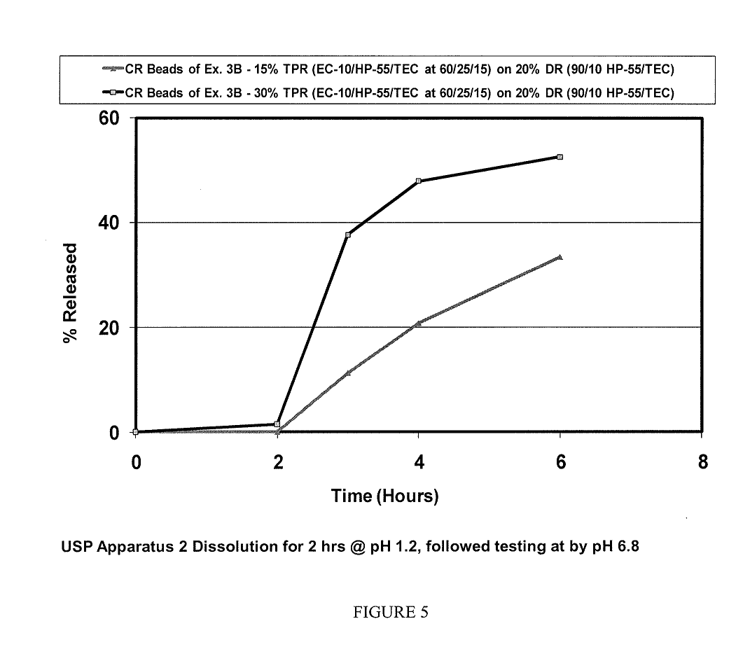

FIG. 5 shows the drug release profiles from the CR beads of Example 3.B at 15% and 30% coating of Example 3.B, demonstrating the combined effect of the inner and outer coating layers.

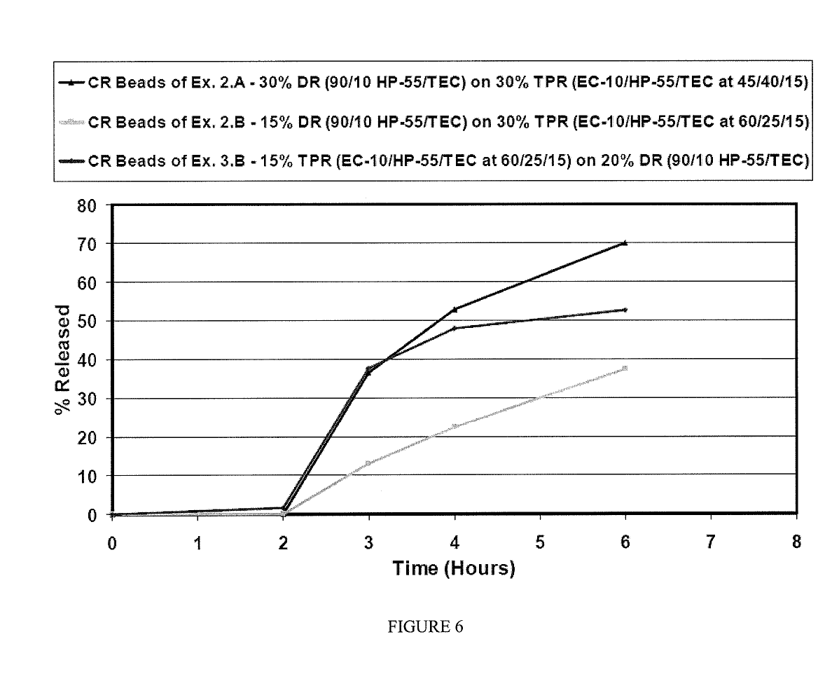

FIG. 6 shows the drug release profiles from the CR beads of the following examples demonstrating the combined effect of the inner and outer coating layers:
CR beads (30% DR-coating (HP-55/TEC at 90/10) on 30% TPR coating (EC-10/HP-55/TEC at 45/40/15)), of Ex. 2.A
CR beads (15% DR-coating (HP-55/TEC at 90/10) on 30% TPR coating (EC-10/HP-55/TEC at 60/25/15)), of Ex. 2.B (see FIG. 3)
CR beads (15% TPR-coating (EC-10/HP-55/TEC at 60/25/15) on 20% DR coating (HP-55/TEC at 90/10)), of Ex. 3.B (see FIG. 4).

### Example 4

### 4.A Pantoprazole IR Beads on Cellets 100 (Drug Load: 20% by Weight)

Pre-dried Cellets 100 (100-200 μm microcrystalline cellulose spheres from Glatt) are sprayed in the Glatt 3 with the pantoprazole solution (25% solids) as described in Example 1.A for a drug load of 20% by weight. The drug-layered beads are also provided with an under-coating of EC-10/Klucel LF/TEC at a ratio of 50/40/10 for a weight gain of 7% as described in Example 1.A, above. The resulting pantoprazole IR beads are sieved to discard oversized particles and fines.

### 4.B CR Beads (30% DR Coating on 30% TPR Coating)

The IR beads (1000 g) from Example 4.A are sprayed with the functional polymer coating formulation, TPR (EC-10/HP-55/TEC at 70/20/10) dissolved in 95/5 acetone/water at 7.5% solids) in the Glatt 3 as described in Example 1.B, above for a weight gain of 30%. These TPR beads at 30% coating (1000 g) are further coated with a functional DR polymer (HP-55/TEC at 90/10) solution in a 75/25 acetone/water mixture at 6% solids for a weight gain of 30% by weight, as described in Example 2. Samples are pulled at 20% and 25% w/w coating for analytical testing (i.e., HPLC assay and drug release). The CR beads with a particle size<355 μm are collected by sieving.

### 4.C Pantoprazole CR Beads (30% TPR Coating on 30% DR Coating)

The IR beads (1000 g) from Example 4.A are first sprayed with a DR coating formulation (HP-55/TEC at 90/10) dissolved in 80/20 acetone/water at 6% solids in the fluid-(Glatt 3) as described in Example 2 for a weight gain of 30%. The DR beads at 30% coating are further coated with a TPR coating solution (EC-10/HP-55/TEC at 70/20/10) dissolved in the 95/5 acetone/water mixture at 7.5% solids for a weight gain of 30%, as described in Example 1.B, above. Samples are also pulled at 20% and 25% w/w coating for analytical testing (i.e., HPLC assay and drug release). The CR beads with a particle size<355 μm are collected by sieving.

### 4.D Pantoprazole DR Beads (HP-55/TEC Coating at 50% w/w)

The IR beads (1000 g) from Example 4.A are first sprayed with a DR coating formulation (HP-55/TEC at 90/10) dissolved in 80/20 acetone/water at 6% solids in the fluid-bed (Glatt 3) as described in Example 2 for a weight gain of 50%. Samples are also pulled at 30%, 40%, and 45% w/w coating for analytical testing (i.e., HPLC assay and drug release). The CR beads with a particle size<355 μm are collected by sieving.

### 4.E Rapidly Dispersing Microgranules

Rapidly dispersing microgranules are prepared following the procedure disclosed in US Patent Application Publication No. U.S. 2003/0215500, published Nov. 20, 2003, the contents of which are hereby incorporated by reference in its entirety for all purposes. Specifically, D-mannitol (152 kg) with an average particle size of approximately 20 μm or less (Pearlitol 25 from Roquette, France) is blended with 8 kg of cross-linked povidone (Crospovidone XL-10 from ISP) in a high shear granulator (GMX 600 from Vector), granulated with purified water (approximately 32 kg), wet-milled using a Comil from Quadro, and finally tray-dried to provide microgranules having an LOD (loss on drying) of less than about 0.8%. The dried granules are sieved and oversize material is again milled to produce rapidly dispersing microgranules with an average particle size in the range of approximately 175-300 μm.

### 4.F Pantoprazole CR ODTs, 40 mg

Table 1 lists the compositions of the orally disintegrating tablets comprising DR beads from Example 4.D and CR beads from Example 4.B or 4.C, each equivalent to 20 mg pantoprazole base. Pharmaceutically acceptable ingredients (i.e., a peppermint flavor (0.5% by weight), a sweetener (sucralose at 0.4%), Crospovidone 5.0%), and microcrystalline cellulose (Avicel® PH101 at 10%) are blended in a V-blender for 15 minutes to produce a homogeneously blended excipient pre-blend. DR beads and CR beads (each equivalent to 20 mg pantoprazole), excipient pre-blend, and rapidly dispersing microgranules are blended in a V-blender for 16 minutes. ODTs comprising pantoprazole sodium (equivalent to 40-mg pantoprazole base) are compressed using a commercial scale Hata tablet press equipped with an externally lubricating Matsui Ex-Lube system to lubricate punches and dies with magnesium stearate prior to each compression under tableting conditions optimized to provide acceptable tableting properties to be suitable for packaging in HDPE bottles—tooling: 16 mm round, flat face, radius edge; compression force: 12-16 kN; mean weight: 1100 mg; mean hardness: ˜20-80 N; and friability: 0.4-0.8%. Pantoprazole ODT CR 40 mg thus produced rapidly disintegrate in the oral cavity creating a non-gritty, easy-to-swallow suspension comprising coated pantoprazole beads, having target release profiles.

TABLE 1
Compositions of Pantoprazole ODT CR, 40-mg

| | ODT CR - A (CR | | ODT CR - B (CR | |
|---|---|---|---|---|
| | Beads of Ex. 4.B & | | Beads of Ex. 4.C & | |
| | DR Beads of Ex. 4.D) | | DR Beads of Ex. 4.D) | |
| | | mg per | | mg per |
| Ingredients | % per ODT | ODT | % per ODT | ODT |
| Pantoprazole CR Beads | 13.91 | 153.0 | 17.0 | 204.0 |
| (Equivalent 20-mg PTP) | | | | |
| Pantoprazole DR Beads | 13.64 | 150.0 | 16.7 | 200.0 |
| (Equivalent 20-mg PTP) | | | | |
| Rapidly Dispersing | 56.95 | 626.5 | 50.8 | 610.0 |
| Granules | | | | |
| MCC - Avicel PH101 | 10.00 | 110.0 | 10.0 | 120.0 |
| Crospovidone CL-M | 5.00 | 55.0 | 5.0 | 60.0 |
| Magnesium stearate | 0.50 | 5.5 | 0.5 | 6.0 |
| Total | 100.0 | 1100.0 | 100.0 | 1200.0 |

### Example 5

### 5.A Pantoprazole IR Beads (Drug Load: 30% w/w)

Pre-dried Cellets 100 (100-200 μm microcrystalline cellulose spheres from Glatt) are sprayed with the pantoprazole solution (25% solids) as described in Example 1.A, above. The drug-layered beads are provided with an under-coating with EC-10/Klucel® LF/TEC by spraying the solution under processing conditions similar to those used for drug layering and dried in the same Glatt unit for 30 min to drive off residual solvents (including moisture). The resulting pantoprazole IR beads are sieved through 35 and 80 mesh screens to discard oversized particles and fines.

### 5.B Pantoprazole CR Beads (30% DR Coating on 30% TPR Coating)

The IR beads (1000 g) from Example 5.A are first coated with a TPR coating formulation (EC-10/HP-55/TEC at 65/25/10) dissolved in 95/5 acetone/water at 7.5% solids) in the fluid-bed coater (Glatt 3) for a gain of 30%, as described in Example 4, above. These TPR beads (1000 g) are further coated a DR solution (HP-55/TEC) for a weight gain of 30%, as described in Example 4. The CR beads with a particle size<420 μm are collected by sieving.

### 5.C Pantoprazole DR Beads (50% w/w)

The IR beads (1000 g) from step 5.A, above are sprayed in the Glatt GPCG 3 with a DR coating formulation (HP-55/TEC at 90/10) dissolved in a 80/20 acetone/water mixture at 6% solids for a gain of 50 wt. %, as described in Ex. 4, above. The DR beads with a particle size<420 μm are collected by sieving.

### 5.D Pantoprazole ODT CR, 80 mg

Pharmaceutically acceptable ingredients (i.e., a peppermint flavor at 0.5% by weight, a sweetener (sucralose) at 0.5% by weight, a colorant such as FD&C Red at 0.3% by weight) are pre-blended with crospovidone and microcrystalline cellulose (Ceolus KG 1000) to obtain a homogenous blend. Appropriate amounts of DR beads from Example 5.C, above and CR beads from Example 5.B, above (each equivalent to 40 mg pantoprazole base), pre-blend, and rapidly dispersing microgranules are blended in a V-blender for 15 minutes to achieve a homogeneously blended compression mix. This compression mix is compressed into ODTs comprising pantoprazole sodium (equivalent to 80 mg pantoprazole base) using a commercial scale rotary tablet press, equipped with an external lubrication system (e.g., a Hata tablet press—Matsui ExLube system) under tableting conditions optimized to provide acceptable tableting properties to be suitable for packaging in HDPE bottles, Aclar 200 blisters with a peel-off paper backing, and/or ‘push-through’ Aclar blister packs. For example, ODTs comprising pantoprazole sodium as DR and CR beads (each equivalent to 40 mg pantoprazole base) are compressed at the following conditions—tooling: 16 mm round, flat face, radius edge; compression force: 12-20 kN; mean weight: 1400 mg; mean hardness: ˜20-80 N; and friability: 0.4-0.8%. Pantoprazole ODT CR 80-mg thus produced rapidly disintegrated in the oral cavity creating a non-gritty, easy-to-swallow suspension comprising coated pantoprazole beads, having target release profiles.

It is to be understood that while the invention has been described in conjunction with specific embodiments thereof, that the description above as well as the examples herein are intended to illustrate and not limit the scope of the invention. Any modification within the scope of the invention will be apparent to those skilled in the art to which the invention pertains.

1. A pharmaceutical composition comprising a first population of controlled-release particles, wherein the controlled-release particles of the first population comprise:
a) a core comprising a proton pump inhibitor or a pharmaceutically acceptable salt, hydrate, polymorph, ester, and/or solvate thereof, and an alkaline agent;
b) a first coating disposed over said core, comprising an enteric polymer; and
c) a second coating disposed over the core, comprising an enteric polymer and a water-insoluble polymer,
wherein said first coating is substantially free of water-insoluble polymers.

2. The pharmaceutical composition of claim 1, wherein said first coating is disposed over said core and said second coating is disposed over said first coating.

3. The pharmaceutical composition of claim 1, wherein said second coating is disposed over said core and said first coating is disposed over said second coating.

4. The pharmaceutical composition of claim 1, wherein said proton pump inhibitor is selected from the group consisting of pantoprazole, omeprazole, esomeprazole, lansoprazole, rabeprazole, pariprazole, lemiprazole, tenatoprazole, nepaprazole, ilaparazole and a pharmaceutically acceptable salt, hydrate, polymorph, ester, and/or solvate thereof

5. The pharmaceutical composition of claim 4, wherein said proton pump inhibitor comprises pantoprazole sodium or a pharmaceutically acceptable hydrate thereof.

6-11. (canceled)

12. The pharmaceutical composition of claim 1, further comprising:
a) at least one sealant layer disposed over said core and under said first and second coatings; and
b) at least one compressible coating disposed over said first and second coatings,
wherein said sealant layer and said compressible coating each comprise a hydrophilic polymer, which may be the same or different.

13. The pharmaceutical composition of claim 12, wherein said at least one sealant layer and said at least one compressible coating independently comprise hydroxypropylcellulose, hydroxypropyl methylcellulose, poly(vinyl acetate-vinyl pyrrolidone), polyvinyl acetate, ethylcellulose, or mixtures thereof.

14. The pharmaceutical composition of claim 9, wherein said compressible coating comprises from about 2% to about 10% by weight of said controlled-release particle.

15. The pharmaceutical composition of claim 1, wherein said enteric polymers in said first and second coatings are each independently selected from the group consisting of hydroxypropyl methyl cellulose phthalate, cellulose acetate phthalate, hydroxypropyl methylcellulose acetate succinate, polyvinyl acetate phthalate, pH-sensitive methacrylic acid-methylmethacrylate copolymers, shellac, and mixtures thereof.

16. The pharmaceutical composition of claim 1, wherein said enteric polymers in said first and second coatings each comprise hydroxypropyl methylcellulose phthalate.

17. The pharmaceutical composition of claim 1, wherein said water-insoluble polymer is selected from the group consisting of ethylcellulose, cellulose acetate, cellulose acetate butyrate, polyvinyl acetate, neutral methacrylic acid-methylmethacrylate copolymers, and mixtures thereof.

18. The pharmaceutical composition of claim 1, wherein said water-insoluble polymer comprises ethylcellulose.

19. The pharmaceutical composition of claim 1, wherein at least one of said first and second coatings further comprises a plasticizer.

20-21. (canceled)

22. The pharmaceutical composition of claim 1, wherein said alkaline agent is selected from the group consisting of sodium carbonate, sodium bicarbonate, sodium hydroxide, monosodium dihydrogen phosphate, disodium hydrogen phosphate, trisodium phosphate, sodium acetate, sodium silicate, magnesium carbonate, magnesium oxide, magnesium hydroxide, magnesium metasilicate aluminate, magnesium silicate aluminate, magnesium silicate, aluminum magnesium hydroxide, magnesium phosphate, magnesium acetate, magnesium carbonate, complex magnesium aluminum metasilicate, calcium carbonate, calcium hydroxide, potassium carbonate, potassium bicarbonate, calcium silicate, monopotassium dihydrogen phosphate, dipotassium hydrogen phosphate, tripotassium phosphate, potassium acetate, and mixtures thereof.

23. The pharmaceutical composition of claim 22, wherein the ratio of said proton pump inhibitor to said alkaline agent ranges from about 7:1 to about 1:3.

24. The pharmaceutical composition of claim 1, wherein said core comprises a buffer layer comprising said alkaline agent.

25. The pharmaceutical composition of claim 1, wherein the ratio of said water-insoluble polymer to said enteric polymer in said second coating ranges from about 10:1 to about 1:4.

26. The pharmaceutical composition of claim 1, wherein the combined weight of said first and second coatings ranges from about 20% to about 70% of said controlled-release particle.

27. The pharmaceutical composition of claim 1, wherein said core comprises a granule, a granulate, a drug crystal, a pellet, a mini-tablet, or an inert bead coated with a drug layer comprising said proton pump inhibitor or a pharmaceutically acceptable salt, polymorph, ester, and/or solvate thereof.

28. The pharmaceutical composition of claim 27, wherein said inert bead comprises sugar, microcrystalline cellulose, lactose, mannitol-microcrystalline cellulose, lactose-microcrystalline cellulose, or silicon dioxide.

29.-31. (canceled)

32. The pharmaceutical composition of claim 27, wherein said drug layer further comprises said alkaline agent.

33. (canceled)

34. The pharmaceutical composition of claim 1, wherein said first population of controlled-release beads provides a lag time of from about 1 hour to about 6 hours, followed by release of said proton pump inhibitor over a period of from about 2 hours to about 6 hours.

35. The pharmaceutical composition of claim 1, further comprising a second population of controlled-release particles, wherein the controlled-release particles of the second population comprise:
a) a second core comprising a proton pump inhibitor or a pharmaceutically acceptable salt, polymorph, solvate, and/or ester thereof, and an alkaline buffer; and
b) at least one controlled-release coating disposed over said core, said at least one controlled-release coating comprising an enteric polymer.

36. The pharmaceutical composition of claim 35, wherein said proton pump inhibitor is selected from the group consisting of pantoprazole, omeprazole, esomeprazole, lansoprazole, rabeprazole, pariprazole, lemiprazole, and/or a pharmaceutically acceptable salt, ester, or solvate thereof.

37. (canceled)

38. The pharmaceutical composition of claim 35, wherein said controlled-release particles of said second population further comprise a sealant layer underlying said enteric coating and/or a compressible coating disposed over said enteric coating, said sealant layer and said compressible coating each independently comprising hydroxypropylcellulose, hydroxypropyl methylcellulose, poly(vinyl acetate-vinyl pyrrolidone), polyvinyl acetate, ethylcellulose, or mixtures thereof.

39.-42. (canceled)

43. The pharmaceutical composition of claim 35, wherein said controlled-release particles of said second population release at least about 75% of said proton pump inhibitor within about 60 minutes when tested for dissolution in USP Apparatus 1 (baskets at 100 rpm) or Apparatus 2 (paddles at 50 rpm) in 900 mL buffer at pH 6.8 at 37° C.

44. The pharmaceutical composition of claim 35, wherein after administration to a patient, said controlled-release particles of said second population provide substantially complete release of said proton pump inhibitor upon entry into the intestine of the patient.

45. The pharmaceutical composition of claim 35, wherein said first and second populations of controlled-release particles exhibit a bimodal pulsatile release profile providing two peaks in blood plasma concentration of said proton pump inhibitor separated by about 1 to about 6 hours.

46. The pharmaceutical composition of claim 35, wherein the controlled-release particles of the second population release said proton pump inhibitor at a substantially different rate compared to the controlled-release particles of the first population.

47. The pharmaceutical composition of claim 35, wherein said first population of controlled-release particles exhibits a lag time of about 1 to about 6 hours, followed by release of said proton pump inhibitor contained therein over a period of about 2 hours to about 6 hours; and wherein said second population of controlled-release particles provides substantially complete release of said proton pump inhibitor contained therein upon entry into the intestine or within about 60 minutes when tested for dissolution in USP Apparatus 1 (baskets at 100 rpm) or Apparatus 2 (paddles at 50 rpm) in 900 mL buffer at pH 6.8 at 37° C.

48. The pharmaceutical composition of claim 35, wherein the ratio of said second population of controlled-release particles to said first population of controlled-release particles ranges from about 25:75 to about 75:25.

49. A pharmaceutical dosage form comprising:
a) the pharmaceutical composition of claim 1; and
b) rapidly dispersing granules comprising a saccharide and/or sugar alcohol in combination with a disintegrant.

50. A pharmaceutical dosage form comprising:
a) the pharmaceutical composition of claim 35; and
b) rapidly dispersing granules comprising a saccharide and/or sugar alcohol in combination with a disintegrant.

51.-53. (canceled)

54. The pharmaceutical composition of claim 49 or 50, wherein said disintegrant and said sugar alcohol and/or said saccharide are each present in the form of microparticles having an average particle size of about 30 μm or less.

55. (canceled)

56. The pharmaceutical dosage form of claim 49 or 50, wherein said dosage form is a capsule or an orally disintegrating tablet.

57.-61. (canceled)

62. A method of preparing the controlled-release particles of claim 1, comprising:
a) preparing a core comprising said proton pump inhibitor or a pharmaceutically acceptable salt, solvate, and/or ester thereof, a polymeric binder, and an alkaline buffer;
b) applying a first coating comprising an enteric polymer; and
c) applying a second coating comprising an enteric polymer in combination with a water-insoluble polymer.

63. The method of claim 62, further comprising:
a) preparing a second core comprising said proton pump inhibitor or a pharmaceutically acceptable salt, polymorph, solvate, and/or ester thereof, a polymeric binder, and a alkaline buffer; and
b) applying an enteric polymer coating to said second core.

64. (canceled)

65. The method of claim 62 or 63, further comprising:
a) mixing said controlled-release particles with rapidly dispersing granules comprising a saccharide and/or sugar alcohol in combination with a disintegrant, thereby forming a compressible blend; and
b) compressing said compressible blend into an orally disintegrating tablet.

66. (canceled)

67. A method of preparing a capsule comprising the controlled-release particles of claim 1 or 35, comprising filling said controlled-release particles into a capsule.

68. (canceled)

69. A method of treating a disease or condition comprising administering to a patient in need thereof a therapeutically effective amount of the composition of claim 1 or 35.

70-71. (canceled)

72. The pharmaceutical composition of claim 65, wherein said first population of controlled-release beads exhibit a drug release profile substantially corresponding to the following pattern when dissolution tested using United States Pharmacopoeia Apparatus 2 (paddles @50 rpm) in a 2-stage dissolution media (700 mL of 0.1N HCl for the first 2 hrs followed by testing in 900 mL buffer at pH 6.8 obtained by adding 200 mL of a pH modifier) at 37° C.:
after 1 hour, no more than about 30% of the total proton pump inhibitor is released;
after 4 hours, from about 30-70% of the total proton pump inhibitor is released; and
after 12 hours, not less than about 60% of the total proton pump inhibitor is released.

73. The pharmaceutical composition of claim 67, wherein said first population of controlled-release beads exhibit a drug release profile substantially corresponding to the following pattern when dissolution tested using United States Pharmacopoeia Apparatus 2 (paddles @50 rpm) in a 2-stage dissolution media (700 mL of 0.1N HCl for the first 2 hrs followed by testing in 900 mL buffer at pH 6.8 obtained by adding 200 mL of a pH modifier) at 37° C.:
after 1 hour, no more than about 30% of the total proton pump inhibitor is released;
after 4 hours, from about 30-70% of the total proton pump inhibitor is released; and
after 12 hours, not less than about 60% of the total proton pump inhibitor is released.
